(19) United States
(12) Patent Application Publication
(10) Pub. No.: US 2025/0312281 A1
(43) Pub. Date: Oct. 9, 2025

(54) SYSTEM FOR CONTROLLABLE RELEASE IN 3D-PRINTED COLON TARGETING (CORR3CT) ORAL DOSAGE FORM

(71) Applicant: CRAFT HEALTH PTE LTD, Singapore (SG)

(72) Inventors: Seng Han LIM, Singapore (SG); Wei Jiang GOH, Singapore (SG); Yi Hsuan OU, Singapore (SG)

(21) Appl. No.: 18/863,575

(22) PCT Filed: Apr. 29, 2023
(86) PCT No.: PCT/IB2023/054483

§ 371 Date: Nov. 6, 2024

Related U.S. Application Data
(60) Provisional application No. 63/338,886, filed on May 6, 2022.

(51) Int. Cl. A61K 9/28; A61K 9/00

(52) U.S. Cl. CPC A61K 9/2893; A61K 9/0053

(57) ABSTRACT

An oral dosage form for controllable release and method of forming are described. The oral dosage form includes a core layer including an active ingredient and a plurality of outer shell layers surrounding the core layer, the plurality of outer shell layers configured to disintegrate at a pH above a threshold. The method of forming the oral dosage form includes forming a core formulation paste including an active ingredient and forming an outer shell formulation paste configured to disintegrate at a pH above a threshold. The method further includes forming a wet tablet by sequentially depositing the outer shell formulation paste and the core formulation paste. The wet tablet is subsequently dried and cooled to produce the oral dosage form.

This application claims the benefit of U.S. Provisional Application No. 63/338,886 filed May 6, 2022, the entire contents of which are hereby incorporated for all purposes in their entirety.

### BACKGROUND OF THE INVENTION

Oral dosage forms are typically designed to release active ingredients within the stomach or small intestines. Few oral dosage forms are engineered to release an encapsulated active ingredient at the large intestines or the colon. This is because the oral dosage form must retain its therapeutic properties through the harsh acidic contents of the stomach and survive the passage of time intact through the small intestine where it can then release the drug content into the colon. Therefore, a universal colon targeting formulation for an oral dosage form may be beneficial to pharmaceutical industries aiming for colon targeting oral dosage form.

In addition, diseases that affect the colon, such as inflammatory bowel disease (IBD) usually have a wide spectrum of clinical phenotypes with either localized and/or diffused inflammation in the gut. Therefore, a substantial portion of IBD patients may benefit from a tunable oral budesonide formulation that can tailor its drug release profile to deliver budesonide to any specific bowel segment (ascending, transverse, and descending).

### BRIEF DESCRIPTION OF THE DRAWINGS

FIG. 1 depicts an example overview of a system for producing an oral dosage form for controllable release, according to at least one example.

FIGS. 2A, 2B, and 2C depict examples of an oral dosage form for controllable release, according to at least one example.

FIG. 3 depicts an example of release profiles of various oral dosage forms, according to at least some embodiments.

FIGS. 4A, 4B, 4C, and 4D depict examples of dissolution profiles and kinetics of multi-layered oral dosage forms.

FIG. 5 depicts an example schematic architecture for implementing techniques relating to generating instructions for manufacturing an oral dosage form, according to at least some embodiments.

FIG. 6 depicts an example of a computing device, according to at least one example.

FIG. 7 depicts a flowchart for an example of a process of forming an oral dosage form for controllable release, according to at least one example.

FIG. 8 depicts a flowchart for another example of a process of forming an oral dosage form for controllable release, according to at least one example.

### DETAILED DESCRIPTION OF THE INVENTION

As described herein, an oral dosage form and method of producing the same is provided to enable an oral dosage form with targeting properties for a particular portion of the gastrointestinal tract, for example the colon. The oral dosage form may be three-dimensionally (3D) printable tablet, mini-tablet, pellet, or capsule with an outer shell. The system of using various 3D printable enteric formulation and different geometrical designs of the outer shell also provides a basis for controlling and extending the rate of drug release in the particular portion of the gastrointestinal tract, thereby, allowing targeting of specific areas within the particular portion of the gastrointestinal tract (e.g., ascending colon, transverse colon, or descending colon), depending on requirement.

In some examples, 3D printing is utilized to 3D print a pill-in-pill configuration or a core and outer shell configuration. Forming the oral dosage form involves configuring a geometric 3D print design, selecting 3D printed materials, and performing the 3D printing. The geometric 3D print design involves the 3D design of a core and outer shell configuration, in the dimensions of a typical oral dosage tablet. The inner core may be supplied as a finished product consisting of oral dosage forms such as tablets, mini-tablets, pellets, or capsules. Alternatively, the inner core may also be formulated as a paste and 3D printed, which can result in significant time-savings when switching between different active pharmaceutical ingredients (APIs) to test for colon targeting effects.

Selecting the 3D printed materials involves the use of a blend of materials in the outer shell formulation. The outer shell formulation can be engineered to remain resistant to the acidic pH of the stomach and break apart when the pH exceeds a threshold (e.g., 7) just before reaching the particular portion of the gastrointestinal tract (e.g., the large intestines), thereby exposing the inner core and releasing the encapsulated APIs. Examples of the main ingredient for the enteric formulation includes, but is not limited to, methyl acrylate-methacrylic acid copolymers, cellulose acetate phthalate, cellulose acetate succinate, hydroxypropyl methylcellulose phthalate, hydroxypropyl methyl cellulose acetate succinate, polyvinyl acetate phthalate, methyl methacrylate-methacrylic acid copolymers, or other enteric polymers.

Performing the 3D printing can involve semi-solid paste extrusion, which does not involve any heat, radiation, or ultraviolet light (UV) curing. The entire process of the 3D printing can be performed at room temperature and pressure. So, the 3D printing can produce a wide variety of therapeutics and APIs, including but not limited to, small molecules, large macromolecules, antibodies, peptides, and proteins. This heat free, radiation free, and UV free technique can reduce the impact of heat, radiation, or UV on the inner core containing the API, mitigating negative influence on stability.

In some examples, with a calculated change in geometric design of the final oral dosage form, the oral dosage form may be altered quickly and predictably, without a change in formulation, to target different parts of the colon, to correspond with the controlled drug release at these specific parts of the colon. This controllability of release profiles can also be achieved by using a different enteric formulation for the outer shell.

As one particular example, the oral dosage form may be a drug delivery system (DDS) to deliver a drug to the large intestine. An example of the drug is budesonide. The DDS may be used for the pharmacological treatment of conditions related to inflammation of the large intestines, including but limited to, inflammatory bowel diseases (IBD), such as Crohn's disease and ulcerative colitis when budesonide is used. These conditions typically involve inflammation of one or more sections of the large intestine. In the interest of clarity of explanation, various embodiments of the present disclosure are described in connection with budesonide as the example drug. However, the embodiments are not limited as such and similarly or equivalently apply to other drugs. Such drugs can be 3D printed for oral ingestion.

As oral budesonide tablets are typically released in the stomach (non-enteric coated tablets) or small intestines (enteric coated tablets), a large proportion of the drug does not reach the targeted site of the large intestine. Taking budesonide via the oral route thus leads to lower localized efficacy and may expose the patient to undesired off-targeting effect.

Instead, budesonide in the form of enemas is often prescribed. Patients are typically advised to use enemas as a route of administration, which is administered through the rectum, and subsequently lie on their side where the large intestine is inflamed to increase contact time of the liquid budesonide with the inflamed section of the large intestine. Enemas are generally intrusive, and as a result patients may not be compliant to the prescribed regimen, leading to sub-optimal therapeutic outcomes. Therefore, the 3D printed oral dosage form seeks to address this through a two-stage release format. The outer shell of the 3D printed oral dosage form that can disintegrate when the pH exceeds a threshold can releases the API at a targeted location, leading to an increased localized concentration of the API at the large intestine.

The oral dosage form can enter the stomach, which has a pH between 1.2 to 1.4, approximately one-hundred twenty minutes after ingestion. The outer shell of the oral dosage form can include an excipient blend of ingredients with enteric properties that can protect the core from exposure to the acidic environment of the stomach. The oral dosage form, with the outer shell still intact, can then enter the small intestines, which has a pH between 5.3 to 7.6, approximately two-hundred eighty-five minutes after ingestion. At the end of the small intestines the pH exceeds 7, which can trigger disintegration of the outer shell to expose the core of the oral dosage form. Then, the oral dosage form can enter the large intestines, which has a pH between 5.9 to 6.0, approximately three-hundred sixty minutes to four-hundred eighty minutes after ingestion. Since the outer shell has disintegrated, the API in the core can be released into the large intestine. In addition, the disintegration rate of the outer shell may be controlled by varying the 3D printed design of the outer shell to target a specific section of the colon, such as the ascending colon, transverse colon, or descending colon.

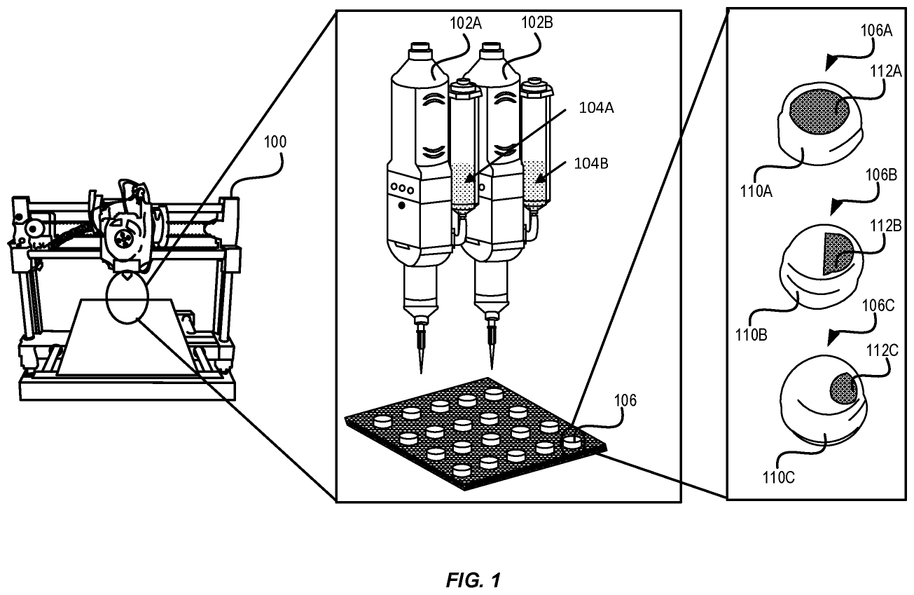

Turning now to the figures, FIG. 1 depicts an example overview of a system for producing an oral dosage form 106 for controllable release, according to at least one example. In the example, the system includes a 3D printer 100 that is used to form an outer shell and a core of the oral dosage form 106. The 3D printer 100 may use semi-solid extrusion because of the lack of heat, radiation, or UV light curing required. This implies that a wide variety of therapeutics and APIs, including but not limited to, small molecules, large macromolecules, antibodies, peptides, and proteins may be utilized. The oral dosage form 106 can be designed as a Computer Aided Design (CAD) file and loaded into the 3D printer 100. The 3D printer 100 may be designed for 3D printing of nutraceuticals and pharmaceuticals, where all wetted areas are medical grade or 316L stainless steel. The 3D printer 100 subsequently 3D prints the oral dosage form 106 as per the CAD design. The CAD design is amenable to change and can be altered for both the outer shell and the core formulation.

3D printer 100 includes two print heads 102, and each of the print heads 102 contain a formulation paste 104 for the oral dosage form 106. For instance, the printing head 102A can include an outer shell formulation paste 104A and the printing head 102B can include a core formulation paste 104B. The core formulation paste 104B can include an API, such as budesonide. Exemplary formulations of the inner core and outer shell are depicted in the Table 1 below. Briefly, the excipients in the specified ratio can be mixed in cold ethanol to form the 3D printable paste.

TABLE 1

| Formulation Type | Formulation | Ranges |
|---|---|---|
| Outer shell | Eudragit S100 | 10% to 30% |
| formulation | PEG6000 | 90% to 70% |
| | | Total: 100% |
| Inner core | Sodium Starch Glycolate (SSG) | 9% to 23% |
| formulation | Polyvinylpyrrolidone (PVP) | 7% to 17% |
| | Mannitol | 4% to 12% |
| | Budesonide | 50% to 90% |
| | Lactose (filler) | q.s. |
| | Deionised water | 20% to 30% of dry weight |
| | | Total: 100% |

The 3D printer 100 can produce various configurations of the oral dosage form 106. For instance, oral dosage form 106A can include an outer shell 110A formed from the outer shell formulation paste 104A and a core 112A formed from the core formulation paste 104B. The core 112A is printed as a whole geometry within the outer shell 110A. Oral dosage form 106B includes an outer shell 110B formed from the outer shell formulation paste 104A and a core 112B formed from the core formulation paste 104B. The core 112B is printed a half geometry within the outer shell 110B. In addition, oral dosage form 106C includes an outer shell 110C formed from the outer shell formulation paste 104A and a core 112C formed from the core formulation paste 104C. The core 112C is printed as a quarter geometry within the outer shell 110C. Other geometries may also be possible. Once 3D printed, the oral dosage form 106 can be left to dry for a time period (e.g., 24 hours) before being ready for use.

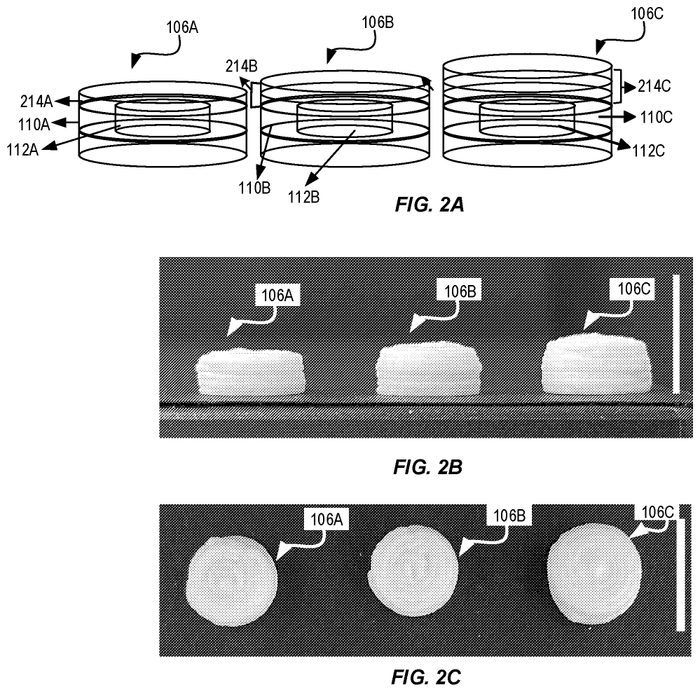

FIGS. 2A-2C depict examples of an oral dosage form 106 for controllable release, according to at least one example. The outer shell 110 of the oral dosage form 106 may have various numbers of top layers 214 over the core 112, resulting in various thicknesses for the oral dosage form 106. The variation in the thickness can serve as a geometric control of the release profile of the API. As illustrated in FIG. 2A, the outer shell 110A of the oral dosage form 106A includes one top layer 214A over the core 112A, the outer shell 110B of the oral dosage form 106B includes two top layers 214B over the core 112B, and the outer shell 110C of the oral dosage form 106C includes three top layers 214C over the core 112C. FIG. 2B illustrates side views of the oral dosage forms 106A-C of FIG. 2A and FIG. 2C illustrates top views of the oral dosage forms 106A-C of FIGS. 2A-2B. The white scale bars in FIGS. 2B and 2C depict 1 cm.

Exemplary properties of oral dosage forms containing budesonide with various layers are described in Table 2.

TABLE 2

| | 1-layer cap | | 2-layer cap | | 3-layer cap | |
|---|---|---|---|---|---|---|
| | Computer | | Computer | | Computer | |
| | Aided | | Aided | | Aided | |
| | Design | Measured | Design | Measured | Design | Measured |
| | (CAD) | values | (CAD) | values | (CAD) | values |
| Weight (mg) | — | 137.85 ± 2.13 | — | 167.08 ± 0.78 | — | 199.35 ± 2.10 |
| Diameter (mm) | 8.87 | 8.72 ± 0.04 | 8.87 | 8.76 ± 0.12 | 8.87 | 8.75 ± 0.11 |
| Height (mm) | 3 | 3.47 ± 0.09 | 3.6 | 3.78 ± 0.09 | 4.2 | 4.46 ± 0.09 |

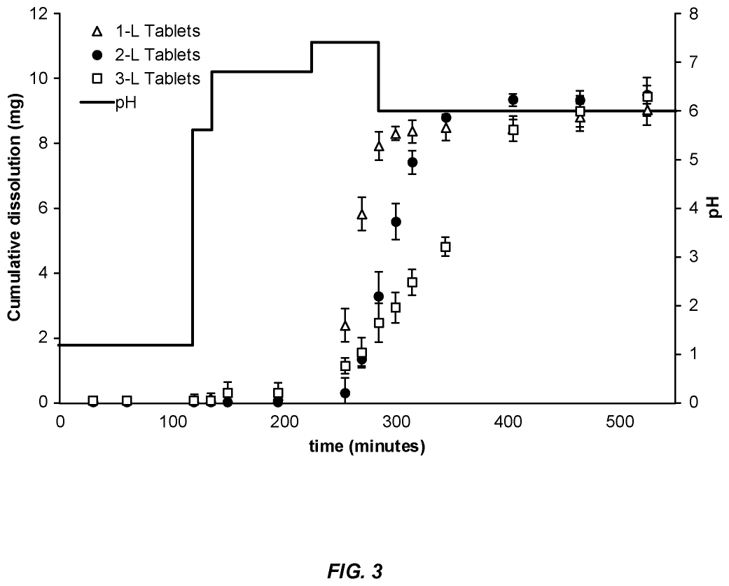

FIG. 3 depicts an example of release profiles of various oral dosage forms, according to at least some embodiments. To mimic the acidic environment of the stomach and subsequent pH changes along the gastrointestinal tract, the oral dosage forms of varying top layer thickness can be added to a pH 1.2 acidic buffer in a dissolution apparatus kept at 37° C. for 2 hours. Subsequently, the pH can be adjusted to correspond to different sections of the gastrointestinal environment. As observed in FIG. 3, all three oral dosage form designs release the API budesonide predominately after pH 7, which corresponds to the large intestines. This demonstrates colon targeting in a surrogate gastrointestinal system. Furthermore, the number of top layers is inversely related to the rate of budesonide release, demonstrating a tunable controlled release of budesonide, which could be used to target different parts of the large intestine.

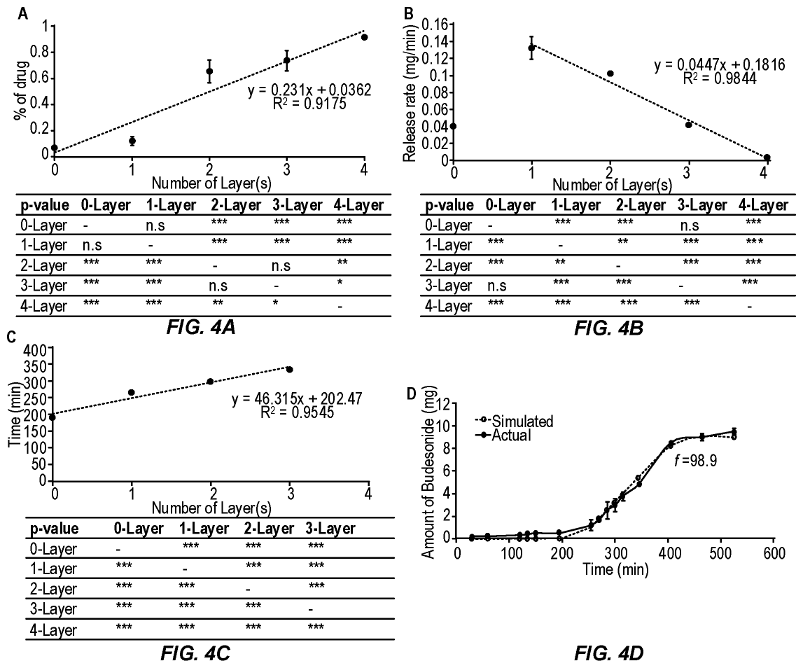

FIGS. 4A-4D depicts an example of dissolution profiles and kinetics of multi-layered oral dosage forms. FIG. 4A shows the relationship between the number of top layers (e.g., top layers 214 in FIG. 2) of the outer shell (e.g., outer shell 110 in FIGS. 1-2) and the percentage of budesonide targeting the colon, FIG. 4B shows the relationship between the number of top layers 214 of the outer shell 110 and the fastest rate of release, and FIG. 4C shows the relationship between the number of top layers 214 of the outer shell 110 and the time taken to reach 50% of budesonide dissolution. A linear relationship is noted (R²=0.9175 for the percentage of drugs targeting the colon; R²=0.9844 for the fastest rate of budesonide release; R²=0.9545 for the time taken to reach 50% of budesonide dissolution) indicating that the release rate of budesonide is directly proportional to the number of top layers 214 (of the outer shell 110) and the dissolution time and amount of budesonide available for colon targeting can be predicted. The rate of budesonide dissolution can be used to generate a simulated drug dissolution profile, with the assumption that the dissolution only occurs after the erosion of the outer shell 110 from 255 minutes (simulated ileum) onwards. FIG. 4B shows the fastest rate of budesonide release is associated with zero top layers, but zero top layers was excluded from the analysis as it is practically does not have a “pill-in-pill” design, since it has its inner core directly exposed to the external medium, the dissolution of 0-layer tablets is most likely governed by the dissolution of R15M solely, rather than the pill-in-pill configuration (core-shell structure). Similarly, in FIG. 4C, time taken for a 4-layer oral dosage form to reach 50% of drug dissolution was excluded in the analysis, due to the fact that 4-layer oral dosage forms did not fully release their budesonide load at the end of the experiment (<30% was released). Since the shell of 4-layer oral dosage tablet was made of RC7000 that dissolves in pH>7, it is possible that the short transit time in simulated ileum (pH7.4) was not sufficient to compromise the thick top layer (in the 4-layer oral dosage form) for the dissolution medium to interact with the inner core, thus there was a slow release of budesonide from the inner core.

FIG. 4D shows an amount of budesonide release at the end of gastric residence. As illustrated, the in vitro profile dissolution of budesonide agrees well with the simulated release profile with a high similarity factor (f2=99). This observation highlights that the drug release profile of budesonide from pill-in-pill 3D printed tablets may be correlated and predictable. The predictability of 3D technology can serve as an effective tool for generating a desired drug release profile and thus facilitate and accelerate product development.

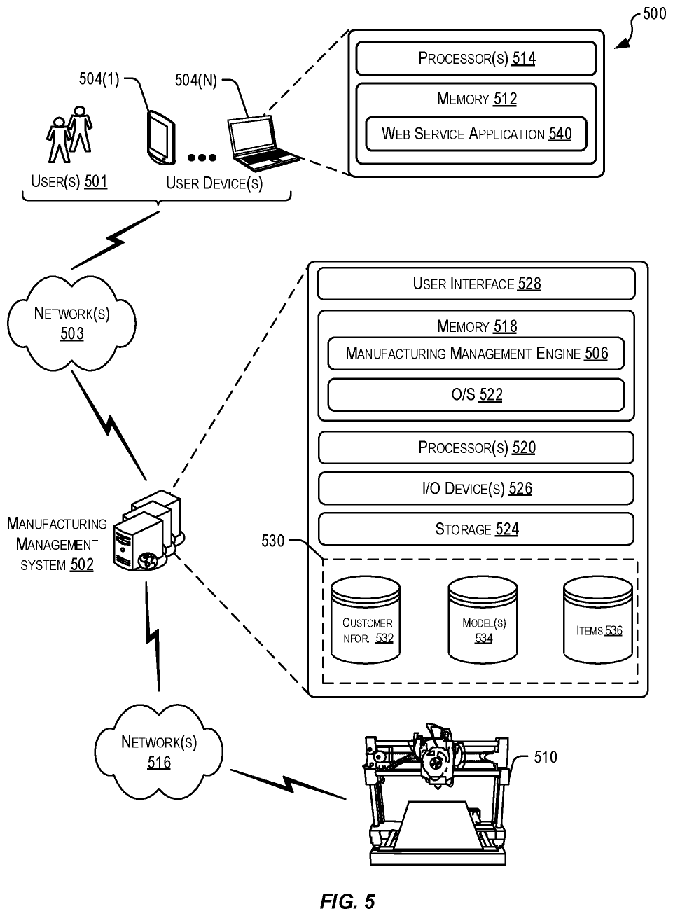

FIG. 5 depicts an example schematic architecture 500 for implementing techniques relating to generating instructions for manufacturing an oral dosage form, according to at least some embodiments. The architecture 500 may include a manufacturing management system 502 in communication with one or more user devices 504(1)-504(N) (hereinafter, “the user device 504”) via one or more networks 503 (hereinafter, “the network 503”). The manufacturing management system 502 communicates with a user device 504 and a production apparatus 510. Using any suitable software, application, etc. running on the user device 504 or otherwise, a user 501 may provide input to the manufacturing management system 502 to design an oral dosage form (e.g., oral dosage form 106 in FIGS. 1-2). The design of the oral dosage form 106 may include the size, shape, number of layers, thickness of layers, types of core formulation paste, types of outer shell formulation paste, types of active ingredients, and other such design factors described herein. The user device 504 may be operable by one or more users 501 (hereinafter, “the user 501”) to interact with the manufacturing management system 502. The network 503 may include any one or a combination of many different types of networks, such as cable networks, the Internet, wireless networks, cellular networks, and other private and/or public networks. The user 501 may be any suitable user including, for example, customers of an electronic marketplace that are associated with the manufacturing management system 502, or any other suitable user.

The production apparatus 510 may include any suitable additive and/or subtractive manufacturing apparatus configured to perform any suitable manufacturing process. For example, the production apparatus 510 is illustrated as an extrusion deposition type of apparatus such as a 3D printer. Other suitable manufacturing apparatuses may be configured to perform processes including, for example, a screen printing machine, a digital ink jet printing machine, a flexo printing machine, a ultra violet (UV) lithography printing machine, laser printing machine, a pad printing machine, a laminated object manufacturing machine, a stereolithography machine, and/or any other suitable additive and/or subtractive production machine. Additional methods and apparatuses for manufacturing may be used in some examples including vacuum forming, thermoplastic forming, casting, injection molding, molding, and the like.

The architecture 500 may also include the production apparatus 510 in communication with at least the manufacturing management system 502 via a secondary network 516. The secondary network 516 may include any one or a combination of many different types of networks as described elsewhere herein.

Turning now to the details of the user device 504, the user device 504 may be any suitable type of computing device such as, but not limited to, a tablet, a mobile phone, a smart phone, a personal digital assistant (PDA), a laptop computer, a desktop computer, a cloud computing device, or any other suitable device capable of communicating with the manufacturing management system 502 via the network 503 or any other suitable network. For example, the user device 504(1) is illustrated as an example of a smart phone, while the user device 504(N) is illustrated as an example of a laptop computer.

The user device 504 may include a web service application 540 within memory 512. Within the memory 512 of the user device 504 may be stored program instructions that are loadable and executable on processor(s) 514, as well as data generated during the execution of these programs. Depending on the configuration and type of user device 504, the memory 512 may be volatile (such as random access memory (RAM)) and/or non-volatile (such as read-only memory (ROM), flash memory, etc.). The web service application 540, stored in the memory 512, may allow the user 501 to interact with the manufacturing management system 502 via the network 503. Such interactions may include, for example, interacting with user interfaces provided by the manufacturing management system 502, selecting oral dosage form 106 designs, customizing oral dosage form 106 capsule designs (e.g., by adjusting a size, thickness, or components within each of the layers), and placing orders for oral dosage forms 106, performing any other interaction described herein or relating to obtaining forms, and any other suitable client-server interactions. The manufacturing management system 502, whether associated with the electronic marketplace or not, may host the web service application 540.

The manufacturing management system 502 may include one or more service provider computers, and may host web service applications. These servers may be configured to host a website (or combination of websites) viewable on the user device 504 (e.g., via the web service application 540). The user 501 may access the website to view items (e.g., capsules) that can be ordered from the manufacturing management system 502 (or an electronic marketplace associated with the manufacturing management system 502). These may be presentable to the user 401 via the web service applications.

The manufacturing management system 502 may include at least one memory 418 and one or more processing units (or processor(s)) 520. The processor 520 may be implemented as appropriate in hardware, computer-executable instructions, software, firmware, or combinations thereof. Computer-executable instruction, software, or firmware implementations of the processor 520 may include computer-executable or machine-executable instructions written in any suitable programming language to perform the various functions described. The memory 518 may include more than one memory and may be distributed throughout the manufacturing management system 502. The memory 518 may store program instructions that are loadable and executable on the processor(s) 520, as well as data generated during the execution of these programs. Depending on the configuration and type of memory including the manufacturing management system 502, the memory 518 may be volatile (such as random access memory (RAM)) and/or non-volatile (such as read-only memory (ROM), flash memory, or other memory). The memory 518 may include an operating system 522 and one or more application programs, modules, or services for implementing the techniques described herein including at least a manufacturing management engine 506. In some examples, the production apparatus 510 is configured to perform the techniques described herein with reference to the manufacturing management system 502, including the manufacturing management engine 506.

The manufacturing management system 502 may also include additional storage 524, which may be removable storage and/or non-removable storage including, but not limited to, magnetic storage, optical disks, and/or tape storage as well as private or public cloud networks. The disk drives and their associated computer-readable media may provide non-volatile storage of computer-readable instructions, data structures, program modules, and other data for the computing devices. The additional storage 524, both removable and non-removable, are examples of computer-readable storage media. For example, computer-readable storage media may include volatile or non-volatile, removable or non-removable media implemented in any suitable method or technology for storage of information such as computer-readable instructions, data structures, program modules, or other data. As used herein, modules, engines, and components, may refer to programming modules executed by computing systems (e.g., processors) that are part of the manufacturing management system 502, the user device 504, and/or the production apparatus 510.

The manufacturing management system 502 may also include input/output (I/O) device(s) and/or ports 526, such as for enabling connection with a keyboard, a mouse, a pen, a voice input device, a touch input device, a display, speakers, a printer, or other I/O device.

The manufacturing management system 502 may also include a user interface 528. The user interface 528 may be utilized by an operator or one of the users 501 to access portions of the manufacturing management system 502. In some examples, the user interface 528 may include a graphical user interface, web-based applications, programmatic interfaces such as application programming interfaces (APIs), or other user interface configurations. The manufacturing management system 502 may also include a data store 530. In some examples, the data store 530 may include one or more data stores, databases, data structures, or the like for storing and/or retaining information associated with the manufacturing management system 502. Thus, the data store 530 may include databases, such as a customer information database 532, a model database 534, and a content item database 536.

The customer information database 532 may be used to retain information pertaining to customers of the manufacturing management system 502, such as the user 501. Such information may include, for example, customer account information (e.g., electronic profiles for individual users), demographic information for customers, payment instrument information for customers (e.g., credit card, debit cards, bank account information, and other similar payment processing instruments), account preferences for customers, shipping preferences for customers, purchase history of customers, oral dosage form models, customer material preferences, and other similar information pertaining to a particular customer and sets of customers of the manufacturing management system 502. In some examples, the customer information may be encrypted and decrypted when needed using typical encryption techniques. In some examples, the customer information may be de-identified or anonymized and instead merely present generic profiles that can be selected from for manufacturing. In some examples, the information retained in the customer information database 532 may be shared with and/or received from the electronic marketplace.

The model database 534 may be used to store three-dimensional models or designs of oral dosage forms 106. The model database 534 may be referenced when the manufacturing management engine 506 attempts to identify a particular three-dimensional item or a particular oral dosage form design, or generate manufacturing instructions for a particular form. The model database 534 may be configured to store any suitable data in any suitable format (e.g., computer-aided drafting (CAD) file such as a STereoLithography file or. STL format) capable of storing a representation of a three-dimensional item.

The digital content item database 536 may be used to retain information about digital content items for which oral dosage form designs are available. For example, the digital content item database 536 may include a table that includes all digital content items available for purchase in the electronic marketplace, information about the design of the different oral dosage forms such as the active ingredients included and the dosage.

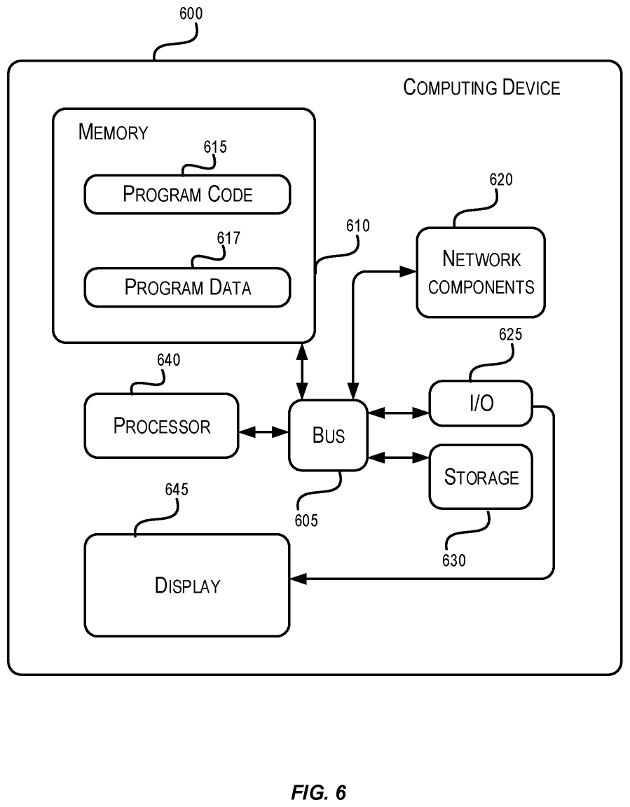

Any suitable computing system or group of computing systems can be used for performing the operations or methods described herein. FIG. 6 depicts an example of a computing device 600. In an embodiment, a computing device, such as user device 504 or manufacturing management system 502 combines the one or more operations and data stores depicted as separate subsystems herein.

FIG. 6 illustrates a block diagram of an example of a computing device 600. Computing device 600 can be any of the described computers herein including, for example, user device 504 or manufacturing management system 502. The computing device 600 can be or include, for example, an integrated computer, a laptop computer, desktop computer, tablet, server, or other electronic device.

The computing device 600 can include a processor 640 interfaced with other hardware via a bus 605. A memory 610, which can include any suitable tangible (and non-transitory) computer readable medium, such as RAM, ROM, EEPROM, or the like, can embody program components (e.g., program code 615) that configure operation of the computing device 600. Memory 610 can store the program code 615, program data 617, or both. In some examples, the computing device 600 can include input/output (“I/O”) interface components 625 (e.g., for interfacing with a display 645, keyboard, mouse, and the like) and additional storage 630.

The computing device 600 executes program code 615 that configures the processor 640 to perform one or more of the operations described herein. The program code 615 may be resident in the memory 610 or any suitable computer-readable medium and may be executed by the processor 640 or any other suitable processor.

The computing device 600 may generate or receive program data 617 by virtue of executing the program code 615. For example, oral dosage form designs, drug characteristics, formulations, and patient treatment profiles are all examples of program data 617 that may be used by the computing device 600 during execution of the program code 615.

The computing device 600 can include network components 620. Network components 620 can represent one or more of any components that facilitate a network connection. In some examples, the network components 620 can facilitate a wireless connection and include wireless interfaces such as IEEE 802.11, Bluetooth, or radio interfaces for accessing cellular telephone networks (e.g., a transceiver/antenna for accessing CDMA, GSM, UMTS, or other mobile communications network). In other examples, the network components 620 can be wired and can include interfaces such as Ethernet, USB, or IEEE 1394.

Although FIG. 6 depicts one computing device 600 with a single processor 640, the system can include any number of computing devices 600 and any number of processors 640. For example, multiple computing devices 600 or multiple processors 640 can be distributed over a wired or wireless network (e.g., a Wide Area Network, Local Area Network, or the Internet). The multiple computing devices 600 or multiple processors 640 can perform any of the steps of the present disclosure individually or in coordination with one another.

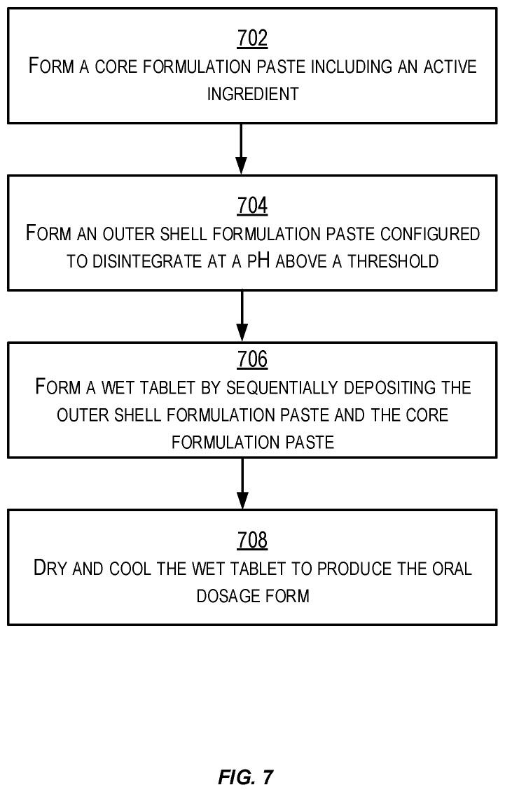

FIG. 7 depicts a flowchart for an example of a process of forming an oral dosage form for controllable release, according to at least one example. In an example, the process involves operation 702, where a core formulation paste (e.g., core formulation paste 104B in FIG. 1) including an active ingredient is formed. The core formulation paste 104B may be formed by mixing the active ingredient with the excipients in the specified ratio in cold ethanol. The core formulation paste 104B can be 3D printable.

In an example, the process includes operation 704, where an outer shell formulation paste (e.g., outer shell formulation paste 104A in FIG. 1). The outer shell formulation paste 104A can be configured to disintegrate at a pH above a threshold (e.g., 7). The threshold can correspond to a pH at a particular region of the gastrointestinal tract, such as the large intestines. The outer shell formulation paste 104A may be formed by mixing components of the outer shell in the specified ratio in cold ethanol. The outer shell formulation paste 104A can be 3D printable.

In an example, the process includes operation 706, where a wet tablet is formed by sequentially depositing the outer shell formulation paste 104A and the core formulation paste 104B. The outer shell formulation paste 104A and the core formulation paste 104B can be sequentially deposited by a 3D printer (e.g., 3D printer 100 in FIG. 1) using semi-solid extrusion. The 3D printer 100 may deposit a bottom layer of the outer shell formulation paste 104A, then middle layers than include both the outer shell formulation paste 104A and the core formulation paste 104B, where the core formulation paste 104A is encapsulated by the outer shell formulation paste 104A. Then, the 3D printer 100 can deposit top layers (e.g., top layers 214 in FIG. 2) of the outer shell formulation paste 104A to fully encapsulate the core formulation paste 104A. The number of top layers 214 can impact a release profile of the active ingredient as the outer shell disintegrates.

In an example, the process includes operation 708, where the wet tablet is dried and cooled to produce the oral dosage form (e.g., oral dosage form 106 in FIGS. 1-2). The drying of the oral dosage form 106 removes any excess water or moisture from the layers that may have been added to formulate the outer shell formulation paste 104A or the core formulation paste 104B. The cooling adds rigidity and hardens the oral dosage form 106. Once dried and stored in a low temperature environment, the oral dosage forms become more stable and durable for storage and transportation.

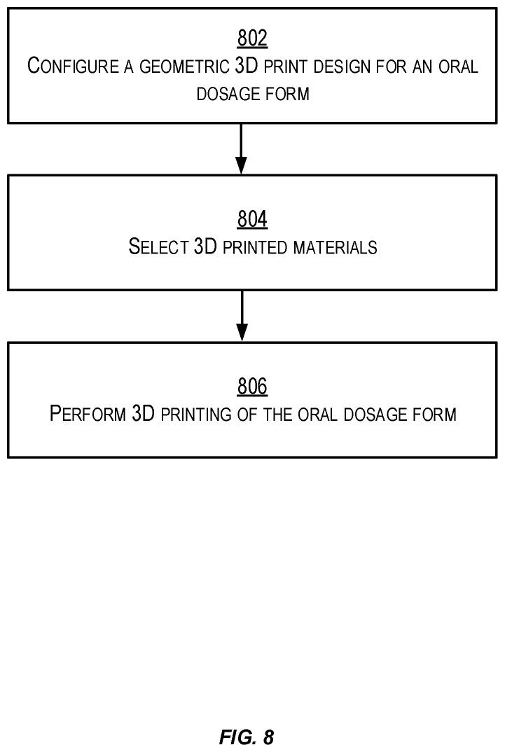

FIG. 8 depicts a flowchart for another example of a process of forming an oral dosage form for controllable release, according to at least one example. In an example, the process includes operation 802, where a geometric 3D print design for an oral dosage form (e.g., oral dosage form 106 in FIGS. 1-2) is configured. The geometric 3D print design involves the 3D design of a core (e.g., core 112 in FIGS. 1-2) and outer shell (e.g., outer shell 110 in FIGS. 1-2) configuration, in the dimensions of a typical oral dosage form. The geometric 3D print design may be configured as a CAD file. The inner core 112 may be supplied as a finished product consisting of oral dosage forms such as tablets, mini-tablets, pellets, or capsules. Alternatively, the inner core 112 may also be formulated as a paste and 3D printed, which can result in significant time-savings when switching between different APIs to test for colon targeting effects. The oral dosage form 106 may be altered quickly and predictably, without a change in formulation, to target different parts of the gastrointestinal tract and to correspond with the controlled drug release at these specific parts of the colon. For instance, a geometry of the core 112 within the outer shell 110 may be configured as part of the geometric 3D print design. The core 112 may have a whole geometry, a half geometry, a quarter geometry, or another geometry within the outer shell 110. In addition, a number of top layers (e.g., top layers 214 in FIG. 2) of the outer shell 110 may be configured as part of the geometric 3D print design based on a desired release profile. This controllability of release profiles can also be achieved by using a different enteric formulation for the outer shell 110.

In an example, the process includes operation 804, where 3D printed materials are selected. Selecting the 3D printed materials can involve using a blend of materials in an outer shell formulation paste (e.g., outer shell formulation paste 104A in FIG. 1). The outer shell formulation paste 104A can be engineered to remain resistant to the acidic pH of the stomach and break apart when the pH exceeds a threshold (e.g., 7) just before reaching the particular portion of the gastrointestinal tract (e.g., the large intestines), thereby exposing the inner core (e.g., core 112 in FIGS. 1-2) and releasing the encapsulated APIs. Examples of the ingredients for the outer shell formulation paste 104A include, but are not limited to, methyl acrylate-methacrylic acid copolymers, cellulose acetate phthalate, cellulose acetate succinate, hydroxypropyl methylcellulose phthalate, hydroxypropyl methyl cellulose acetate succinate, polyvinyl acetate phthalate, methyl methacrylate-methacrylic acid copolymers, or other enteric polymers.

In an example, the process includes operation 806, where the 3D printing of the oral dosage form 106 is performed. Performing the 3D printing can involve a 3D printer (e.g., 3D printer 100 in FIG. 1) performing semi-solid paste extrusion, which does not involve any heat, radiation, or UV curing. The entire process of the 3D printing can be performed at room temperature and pressure. The 3D printer 100 can receive the CAD file specifying the geometric 3D print design for the oral dosage form 106. So, the 3D printing can produce oral dosage forms 106 with a wide variety of therapeutics and APIs, including but not limited to, small molecules, large macromolecules, antibodies, peptides, and proteins. This heat free, radiation free, and UV free technique can reduce the impact of heat, radiation, or UV on the inner core containing the API, mitigating negative influence on stability. The 3D printing can involve forming a wet tablet by sequentially depositing the outer shell formulation paste 104A and a core formulation paste (e.g., core formulation paste 104B in FIG. 1) that includes an active ingredient, such as budesonide. The wet tablet can then be dried and cooled to produce the oral dosage form 106.

Various embodiments of the present disclosure are described in the following examples. Example 1 includes a method of forming an oral dosage form for controllable release targeting a human colon region, the method comprising: forming a core formulation paste including an active ingredient; forming an outer shell formulation paste configured to disintegrate at a pH above a threshold; forming a wet tablet by sequentially depositing the outer shell formulation paste and the core formulation paste; and drying and cooling the wet tablet to produce the oral dosage form.

Example 2 includes the method of example 1, wherein the outer shell formulation paste and the core formulation paste are sequentially deposited using a three dimensional (3D) printer.

Example 3 includes the method of example 2, wherein the 3D printer is configured use semi-solid extrusion to form a set of outer shells using the outer shell formulation paste and a core using the core formulation paste.

Example 4 includes the method of any examples 1-3, wherein forming the wet tablet comprises forming a core printed by using the core formulation paste and forming a set of outer shells printed by using the outer shell formulation paste.

Example 5 includes the method of example 4, wherein an outer shell of the set is configured to disintegrate at a disintegration rate such that the core is targeted released within a section of a colon.

Example 6 includes the method of example 5, wherein a configuration of the outer shell comprises a thickness of the outer shell defined based on the disintegration rate.

Example 7 includes the method of any of examples 4-6, wherein the core is contained within a first outer shell.

Example 8 includes the method of example 7, wherein the wet tablet further comprises a second outer shell printed as a first top layer relative to the first outer shell.

Example 9 includes the method of example 8, wherein the wet tablet further comprises a third outer shell printed as a second top layer relative to the second outer shell.

Example 10 includes the method of any of examples 4-9, wherein the core is contained within a first outer shell, and wherein the wet tablet further comprises a number of layers printed as outer shells by using the outer shell formulation paste.

Example 11 includes an oral form for controllable release targeting a human colon region, comprising: a core including an active ingredient; and a number of outer shells surrounding the core and formed by using a three dimensional (3D) printer, each outer shell configured to disintegrate at a pH above a threshold.

Example 12 includes the oral dosage form of example 11, wherein the active ingredient comprises budesonide.

Example 13 includes the oral dosage form of example 11 or 12, wherein the core is formed by at least 3D printing using a core formulation paste that includes the active ingredient, and wherein the number of outer shells is printed by using an outer shell formulation paste configured to disintegrate at the pH above the threshold.

Example 14 includes the oral dosage form of example 13, wherein an outer shell of the of the number of outer shells is configured to disintegrate at a disintegration rate such that the core is targeted released within a section of a colon.

Example 15 includes the oral dosage form of example 14, wherein a configuration of the outer shell comprises a thickness of the outer shell defined based on the disintegration rate.

Example 16 includes the oral dosage form of any of examples 13-15, wherein the core is contained within a first outer shell.

Example 17 includes the oral dosage form of example 16 further comprising a second outer shell printed as a first top layer relative to the first outer shell.

Example 18 includes the oral dosage form of example 17 further comprising a third outer shell printed as a second top layer relative to the second outer shell.

Example 19 includes the oral dosage form of any of examples 13-18, wherein the core is contained within a first outer shell, and wherein the oral dosage form further comprises a number of layers printed as outer shells by using an outer shell formulation paste.

Example 20 includes the oral dosage form of example 19, wherein the core, the first outer shell, and the number of layers are sequentially printed using semi-solid extrusion.

While the present subject matter has been described in detail with respect to specific aspects thereof, it will be appreciated that those skilled in the art, upon attaining an understanding of the foregoing, may readily produce alterations to, variations of, and equivalents to such aspects. Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter. Accordingly, the present disclosure has been presented for purposes of example rather than limitation, and does not preclude the inclusion of such modifications, variations, and/or additions to the present subject matter as would be readily apparent to one of ordinary skill in the art.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

Aspects of the methods disclosed herein may be performed in the operation of such computing devices. The system or systems discussed herein are not limited to any particular hardware architecture or configuration. A computing device can include any suitable arrangement of components that provide a result conditioned on one or more inputs. Suitable computing devices include multi-purpose microprocessor-based computer systems accessing stored software that programs or configures the computing system from a general purpose computing apparatus to a specialized computing apparatus implementing one or more aspects of the present subject matter. Any suitable programming, scripting, or other type of language or combinations of languages may be used to implement the teachings contained herein in software to be used in programming or configuring a computing device. The order of the blocks presented in the examples above can be varied—for example, blocks can be re-ordered, combined, and/or broken into sub-blocks. Certain blocks or processes can be performed in parallel.

What is claimed is:

1. A method of forming an oral dosage form for controllable release targeting a human colon region, the method comprising:
forming a core formulation paste including an active ingredient;
forming an outer shell formulation paste configured to disintegrate at a pH above a threshold;
forming a wet tablet by sequentially depositing the outer shell formulation paste and the core formulation paste; and
drying and cooling the wet tablet to produce the oral dosage form.

2. The method of claim 1, wherein the outer shell formulation paste and the core formulation paste are sequentially deposited using a three dimensional (3D) printer.

3. The method of claim 2, wherein the 3D printer is configured use semi-solid extrusion to form a set of outer shells using the outer shell formulation paste and a core using the core formulation paste.

4. The method of claim 1, wherein forming the wet tablet comprises forming a core printed by using the core formulation paste and forming a set of outer shells printed by using the outer shell formulation paste.

5. The method of claim 4, wherein an outer shell of the set is configured to disintegrate at a disintegration rate such that the core is targeted released within a section of a colon.

6. The method of claim 5, wherein a configuration of the outer shell comprises a thickness of the outer shell defined based on the disintegration rate.

7. The method of claim 4, wherein the core is contained within a first outer shell.

8. The method of claim 7, wherein the wet tablet further comprises a second outer shell printed as a first top layer relative to the first outer shell.

9. The method of claim 8, wherein the wet tablet further comprises a third outer shell printed as a second top layer relative to the second outer shell.

10. The method of claim 4, wherein the core is contained within a first outer shell, and wherein the wet tablet further comprises a number of layers printed as outer shells by using the outer shell formulation paste.

11. An oral dosage form for controllable release targeting a human colon region, comprising:
a core including an active ingredient; and
a number of outer shells surrounding the core and formed by using a three dimensional (3D) printer, each outer shell configured to disintegrate at a pH above a threshold.

12. The oral dosage form of claim 11, wherein the active ingredient comprises budesonide.

13. The oral dosage form of claim 11, wherein the core is formed by at least 3D printing using a core formulation paste that includes the active ingredient, and wherein the number of outer shells is printed by using an outer shell formulation paste configured to disintegrate at the pH above the threshold.

14. The oral dosage form of claim 13, wherein an outer shell of the of the number of outer shells is configured to disintegrate at a disintegration rate such that the core is targeted released within a section of a colon.

15. The oral dosage form of claim 14, wherein a configuration of the outer shell comprises a thickness of the outer shell defined based on the disintegration rate.

16. The oral dosage form of claim 13, wherein the core is contained within a first outer shell.

17. The oral dosage form of claim 16 further comprising a second outer shell printed as a first top layer relative to the first outer shell.

18. The oral dosage form of claim 17 further comprising a third outer shell printed as a second top layer relative to the second outer shell.

19. The oral dosage form of claim 13, wherein the core is contained within a first outer shell, and wherein the oral dosage form further comprises a number of layers printed as outer shells by using an outer shell formulation paste.

20. The oral dosage form of claim 19, wherein the core, the first outer shell, and the number of layers are sequentially printed using semi-solid extrusion.
